(19) United States
(12) Patent Application Publication
(10) Pub. No.: US 2011/0202170 A1
(43) Pub. Date: Aug. 18, 2011

(54) ACCESS AND INVENTORY CONTROL FOR CLIMATE CONTROLLED STORAGE

(76) Inventors: Dennis K. Dawes, Indianapolis, IN (US); David L. Helmer, Indianapolis, IN (US); Bruce F. King, Indianapolis, IN (US)

(21) Appl. No.: 13/022,708

(22) Filed: Feb. 8, 2011

Related U.S. Application Data
(60) Provisional application No. 61/302,643, filed on Feb. 9, 2010.

(51) Int. Cl. G06F 17/00

(52) U.S. Cl. 700/215; 700/214

(57) ABSTRACT

An apparatus and method for storing medical products such as pharmaceutical and medical products in climate controlled storage devices includes climate control systems in communication with inventory access and tracking systems. The inventory stored may be accessed by an authorized user providing a barcode input or other authorizing input. The apparatus may log climate parameters and associate the climate parameters with the items in storage. The system may limit access to particular storage locations to provide control of the inventory.

### CROSS-REFERENCE TO RELATED APPLICATIONS

This application claims priority to U.S. Provisional Application Ser. No. 61/302,643, filed Feb. 9, 2010, which is incorporated by reference herein in its entirety.

### BACKGROUND

The present disclosure is related to climate controlled storage systems. More specifically, the present disclosure is related to climate controlled storage systems with integrated inventory tracking and access control functions.

Medical supplies such as pharmaceuticals and blood products are a high value commodity requiring stringent quality and inventory control measures. Medical products including medications, tissues, and blood products such as whole blood, plasma, or platelets, for example, are in limited supply and have a limited shelf life and stringent quality control requirements to maintain the quality of the products. Inventory management of medical products may be accomplished on a distributed basis with a central storage and management provider, such as a blood center, tissue bank, or pharmaceutical distribution center, and multiple use points, such as hospitals, pharmacies, and clinics, covering a broad geographic region. A central entity must, to the extent possible, monitor inventory quantities, age, identity and consumption of products at each of the multiple use points in order to appropriately supply the products and effectively move aging products to a point where the products will be used prior to expiration.

### SUMMARY OF THE INVENTION

The present application discloses one or more of the features recited in the appended claims and/or the following features which, alone or in any combination, may comprise patentable subject matter:

According to one aspect of the present disclosure, a medical products storage device embodied as a refrigerator includes cabinet, a storage space, a door covering the storage space, and a plurality of drawers positioned in the storage space. The refrigerator further includes access control devices which permit a user to access the storage space by permitting the door to be opened. Additional access control devices are operable to permit a user to access one or more of the drawers positioned in the storage space. The refrigerator further includes a controller operable to control a climate control device to control the environment of the storage space. The refrigerator still further includes an interface unit electrically coupled to the controller and operable to receive a signal from the controller to control the operation of the access control devices. In some embodiments, the storage device may be embodied as a freezer while in other embodiments, the storage device may be embodied as an incubator.

In some embodiments, the controller is connected to a barcode reader and controls access to a particular storage location based on a signal from the barcode reader. In some embodiments, the controller is connected to a user interface. The user interface is operable to allow a user to input a request for access to a particular inventory location. In some embodiments, the controller may be connected to an external inventory and access control monitoring system. The refrigerator may be one of several storage devices connected to the external inventory and access control monitoring system with the system controlling access to inventory locations in a plurality of storage devices.

In some embodiments, the controller is operable to receive signals from sensors which provide the controller with measurements of the current climate conditions within the storage space. The controller may be operable to associate the climate information with inventory records to log the storage conditions for a particular item of inventory. The inventory log may be shared with the external inventory and access control monitoring system.

In some embodiments, the controller may permit limited access to certain storage locations based on the identity of the user requesting access. In some embodiments, the user may input a particular item of inventory being requested and the controller may limit access to storage locations that include only the requested inventory item. In some embodiments, the controller may request a user scan an item of inventory being removed with the controller updating the inventory records based on the scanned item data.

In some embodiments, the refrigerator may include radio frequency identification monitoring such that each item of inventory is constantly monitored based on a radio frequency identification signal emitted by a tag associated with the particular item of inventory. The controller may be operable to detect that a particular item of inventory has been removed based on the absence of a radio frequency identification signal. The controller may also be operable to receive a signal from a sensor indicating the status of an inventory location, including whether the inventory location is opened. If the controller detects an unexpected open to condition for a particular inventory location, the controller may log the condition. In addition, the controller may also provide an alarm that an unexpected inventory location has been accessed. The alarm may be a local visual and/or audible alarm or the alarm may be transmitted to the external inventory and access control monitoring system.

In some embodiments, the storage device may further include a monitoring system in conjunction with or in addition to the temperature controller. The monitoring system measures and records temperature and other key variables and notifies users of status. The monitoring system may further include programmable alarm settings to indicate out of tolerance conditions of the key variables. In some embodiments, alarm status and measurement display may be signaled to a user via visual, auditory, text message, phone dialer, or network connection.

In some embodiments, the climate control device cools, chills, or refrigerates the entire storage space. In other embodiments, the climate control device is operable to warm or heat the entire storage space. In some embodiments, the climate control device is a peltier device. In some embodiments, a separate peltier deice is used in each storage location and the controller is operable to maintain each location at a different temperature, with the control of the temperature of a particular storage space being maintained by a climate control device, such as a peltier device.

According to another aspect of the present disclosure, a medical products inventory and quality control system comprises a plurality of medical products storage containers. Each container includes a marking tag configured to transmit and receive wireless signals indicative of parameters associated with the medical products stored in the container. A medical products storage device includes a storage space configured to store the containers. A controller of the storage device is configured to monitor the operational parameters of the device. A wireless transceiver is configured to communicate with the tags of medical product storage containers positioned in the storage space. A data processing circuit is in communication with the controller and the wireless transceiver and is operable to associate the operational parameters of the medical products storage device with the parameters of the medical products stored in each container to create an inventory and quality control record for each container.

In some embodiments the system may further comprise a computer in communication with the data processing circuit, the computer operable to display the inventory and quality control record for each container. The system may further include a plurality of computers in a network, each computer in communication with the data processing circuit of at least one medical products storage device, the network configured to gather inventory and quality control records from a plurality of medical products storage devices.

The marking tag may comprise a processor, a memory device coupled to processor, an RF transceiver coupled to the processor, and a battery coupled to the processor. In some embodiments the tag may further comprise a bar code label. The tag may further comprise an indicator configured to indicate a status of the contents of the storage container associated with the tag. The indicator may comprise a light emitting diode LED. A green LED may be illuminated when the contents of the storage container are safe to use. A yellow LED may be illuminated when the contents of the storage container are nearing the end of their shelf life. A red LED may be illuminated when the contents of the storage container are unsafe to use. In some embodiments, the green LED flashes when the storage container is identified by the system to be used.

In some embodiments, the storage container further comprises a body and the tag is coupled to the body through a coupler. The coupler may be configured to enable the tag to function when the tag is coupled to the body. The tag may be disabled if the coupler is removed. The tag may enter an error mode if the coupler is removed. In the error mode, the tag may not illuminate any indicator if the tag is the error mode.

In some embodiments, the system communicates a status of the contents of the container to the tag. The status may be based on the operational parameters of the medical products storage device. The medical products storage device may control a temperature of the storage space. In some embodiments, the status of the containers positioned within a storage space of a medical products device changes if the temperature within the storage space falls outside of acceptable limits.

The medical products storage device may comprise an agitator. The status of the containers positioned within a storage space of a medical products device may change if the agitator speed is not maintained properly. For example, the status of the containers positioned within a storage space of a medical products device may change if the agitator fails to operate for a predetermined period. Also, the status of the containers positioned within a storage space of a medical products device may change if the agitator speed exceeds an acceptable level.

In some embodiments, the wireless transceiver of the medical products storage device is configured to continuously communicate with the tags of the medical products storage containers. In other embodiments, the wireless transceiver of the medical products storage device is configured to communicate with the tags of the medical products storage containers when the containers are inserted or removed from the storage space. The medical products storage device may include a plurality of transceivers, each transceiver associated with a particular storage location within the storage space, and each transceiver configured to communicate only with the containers positioned in the associated storage location.

In some embodiments, the marking tag further comprises a sensor coupled to the processor. The temperature sensor may be operable to sense a storage condition experienced by the associated blood product storage container. Also, the marking tag may be configured to monitor the storage conditions sensed by the associated blood product storage container and log the data over time. The marking tag may transmit the data related to the storage conditions experienced by the associated blood product storage container to the data processing circuit. In some embodiments, the processor of the marking tag may analyze the data related to the storage conditions experienced by the associated blood product storage container and change the status of the storage container if the conditions of the storage container fall outside of acceptable limits. The sensor may be a temperature sensor such as thermocouple, for example. The sensor may be a motion sensor such as an accelerometer, for example.

In some embodiments, the storage device may further include a monitoring system in conjunction with or in addition to the temperature controller. The monitoring system measures and records temperature and other key variables and notifies users of status. The monitoring system may further include programmable alarm settings to indicate out of tolerance conditions of the key variables. In some embodiments, alarm status and measurement display may be signaled to a user via visual, auditory, text message, phone dialer, or network connection.

Additional features, which alone or in combination with any other feature(s), including those listed above and those listed in the claims, may comprise patentable subject matter and will become apparent to those skilled in the art upon consideration of the following detailed description of illustrative embodiments exemplifying the best mode of carrying out the invention as presently perceived.

### BRIEF DESCRIPTION OF THE DRAWINGS

The detailed description particularly refers to the accompanying figures in which:

FIG. 1 is a diagrammatic representation of a climate controlled storage device including access control and inventory management capabilities;

FIG. 2 is a perspective view of a refrigerator including a number of access controlled storage locations;

FIG. 3 is a front view of an embodiment of a storage drawer including an access control system;

FIG. 4 is a top view of the drawer of FIG. 3 with portions removed;

FIG. 5 is a side view of another embodiment of a storage drawer that includes an access control system;

FIG. 6 is a diagrammatic representation of an inventory and quality control system for medical products according to the present disclosure;

FIG. 7 is a block diagram of an embodiment of a local portion of the inventory and quality control system for medical products shown in FIG. 1;

FIG. 8 is a front elevation view of a first embodiment of a medical products container including a radio frequency tag according to the present disclosure;

FIG. 9 is a side elevation view of the embodiment of blood product container shown in FIG. 3;

FIG. 10 is a front elevation view of a second embodiment of a medical products container including a radio frequency tag according to the present disclosure;

FIG. 11 is a side elevation view of the embodiment of blood product container shown in FIG. 5;

FIG. 12 is a front plan view of the tag of FIGS. 8-11 without a coupler; and

FIG. 13 is a block diagram of the electrical system of a radio frequency tag according to the present disclosure.

### DETAILED DESCRIPTION OF THE DRAWINGS

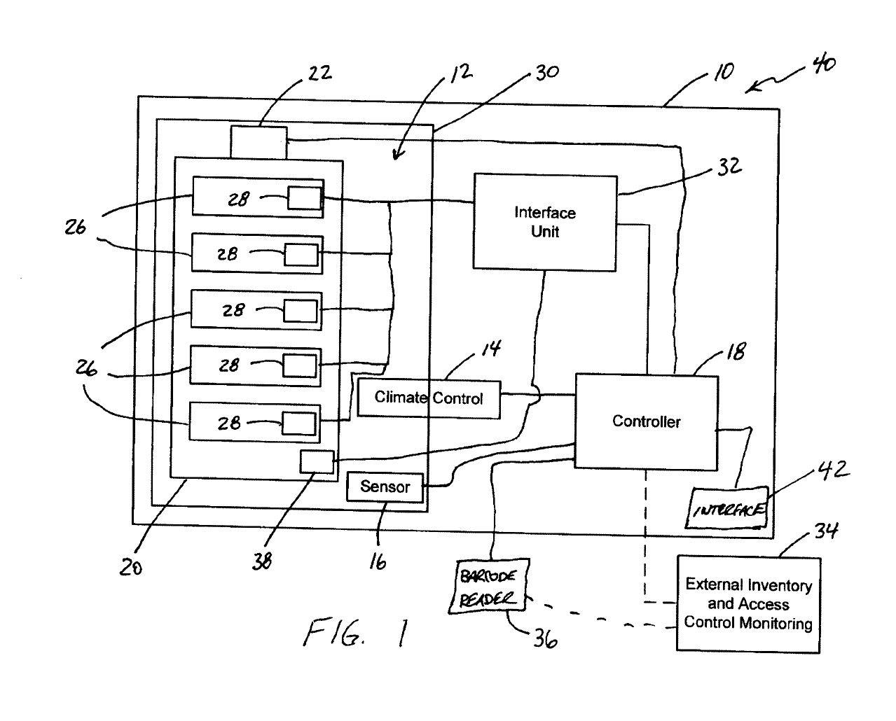

A climate-controlled storage device illustratively embodied as a refrigerator 40 as shown in FIG. 1, includes a cabinet 10 having an enclosure 30 forming a storage space 12 in which a number of storage drawers 26 are positioned. The storage drawers 26 are positioned in the enclosure 30 behind a door 20 which closeable to prevent access to the storage space 12. Each of the drawers 26 are movable between an open position permitting access to the contents of the drawer 26 and a closed position in which the contents of the drawer 26 are not accessible.

A climate control device 14, embodied as a chiller in the embodiment of FIG. 1, is operable to control the temperature of the storage space 12. The temperature in the storage space 12 is monitored by a temperature sensor 38 positioned in the storage space 12. A door status monitor 22 detects whether the door 20 is in an open or closed position.

A controller 18 is electrically connected to the chiller 14, temperature sensor 16, and door status monitor 22. The controller 18 is operable to receive a temperature signal from the sensor 16 and a signal indicative of the status of the door 20 from the monitor 22. The controller 18 is a fully functional processor based control device operable to control climate parameters in the storage space 12 to maintain the storage space 12 climate within acceptable parameters. In the illustrative embodiment, the refrigerator 40 is used to store pharmaceuticals, blood products, or the like. Operation of the refrigerator 40 permits the storage climate to be maintained appropriately for the storage of pharmaceuticals, blood products, and other perishable medical supplies. In some embodiments, the storage device may heat the storage space. In other embodiments, the storage device may be a freezer, for example an ultra low temperature freezer for storing certain biological materials. In some embodiments, the climate control device 14 may also control humidity levels within the storage space 12. In some embodiments, the climate control device 14 is a peltier device. In some embodiments, a separate peltier deice is used in each drawer or other storage location and the controller 18 is operable to maintain each location at a different temperature, with the control of the temperature of a particular storage space being maintained by a separate climate control device 14.

Referring again now to FIG. 1, an interface unit 32 is electrically coupled to the controller 18 and operable to control a number of access control devices 28 which are operable to permit a user to access the respective drawers 26 associated with each of the access control devices 28. The interface unit 32 is also connected to an access control device 38 which is operable to permit or exclude opening of the door 20. As will be described in further detail below, the access control devices 28, 38 may include electromechanical latches which are activated by the interface unit to lock the door 20 or respective drawers 26. The controller 18 is electrically connected to a bar code reader 36 and is operable to receive a signal from the barcode reader 36 and control access to the door 20 and drawers 26 by providing instructions to the interface unit 32. In addition, the controller 18 is in communication with an interface 42 which responds to the controller 18 to provide access to the door 20 and respective drawers 26.

The controller 18 may operate as an inventory tracking and control system so that any materials stored in the refrigerator 40 may only be accessed by qualified users and any inventory transactions are logged by the controller 18 to provide a record showing the storage temperature and other climate parameters for each item in storage. The controller 18 also logs which user removed particular inventory items and when those items were removed. For example, a user may store inventory in the various drawers 26 with each drawer being designated as a particular storage location. A particular user may then be associated with a bar code identification permitting the user to enter an inventory transaction into the controller 18 by scanning a barcode associated with a particular transaction, or by entering a transaction into the user interface 42. The controller 18 processes the inventory transaction request from the user, confirms that the user is permitted to make the inventory transaction, and checks the requested inventory transaction against the inventory that is known to be in the respective drawers 26. The controller also determines the location of the requested inventory, transmits a signal to the interface unit 32 to permit access to the storage space 12 by activating the access control device 38, and provides access to the appropriate drawer 26 by activating the appropriate drawer access control device 28. In some instances, multiple units of similar inventory may be stored in a respective drawer 26. A user may be prompted to scan a label for the inventory being removed across the barcode reader to cross-check that the appropriate inventory is being removed. It should be understood that in some instances, each item of inventory may have a separate serial number identifying both the type and particular unit of inventory. For example, one particular drawer 26 may store five units of blood plasma, with each unit of blood plasma having a unique serial number which is stored in the inventory tracking system of controller 18. Using the barcode reader 36 to scan the label of a particular unit of blood plasma, the controller 18 will log that unique unit of blood plasma being removed by a particular user.

While the controller 18 as described above may operate as an independent inventory tracking system for the particular refrigerator 40, in some instances the controller 18 may be connected to an external inventory and access control monitoring system 34. A system 34 may be linked to multiple storage units besides a particular refrigerator 40. In some embodiments, the controller 18 may maintain the inventory tracking data as described above and communicate that data to the system 34 so that system 34 may operate as a centralized inventory control and access management system. The controller 18 operates as a local inventory control and access system that associates storage conditions with each item of inventory so that the quality, shelf-life, and use of each item of inventory may be associated in a single inventory record. The controller 18 may also be operable to notify the system 34 if the climate in refrigerator 40 falls outside of certain control parameters, if a particular inventory location is accessed improperly, or if a particular inventory location is left open for an extended period.

In some embodiments, the access control devices 28, 38 may include a sensor that detects if a particular drawer 26 or the door 20 is open. In addition, each access control device 28, 30 may include an indicator, such as a light, for example, to indicate the proper inventory location to a user who has requested an inventory transaction. In lieu of an indicator, in some embodiments the access devices 28, 30 automatically open the door 20 or drawers 26 associated with the particular inventory being requested. In some embodiments, each drawer 26 may further include separate and independently accessible bins within each drawer 26, with each bin having a separate access control device similar to an access control device 28, 38 shown in the embodiment of FIG. 1.

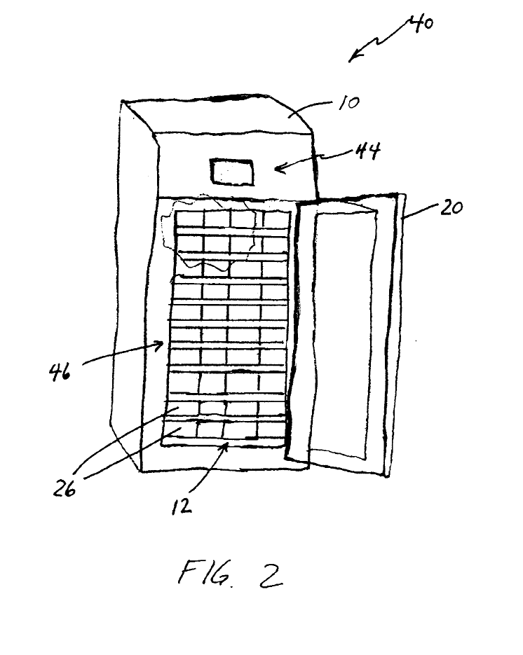

In an implementation of refrigerator 40 shown in FIG. 2, embodiment includes an upright refrigerator cabinet 10 with a door 20 that opens to permit access to the drawers 26. In the embodiment of FIG. 2, the interface 42 includes a display 44 which provides a visual output of various data resident in the controller 18. The refrigerator 40 of FIG. 2 includes a number of drawers 26, with the access control devices 28 integrated into a rack 46 supporting the drawers 26.

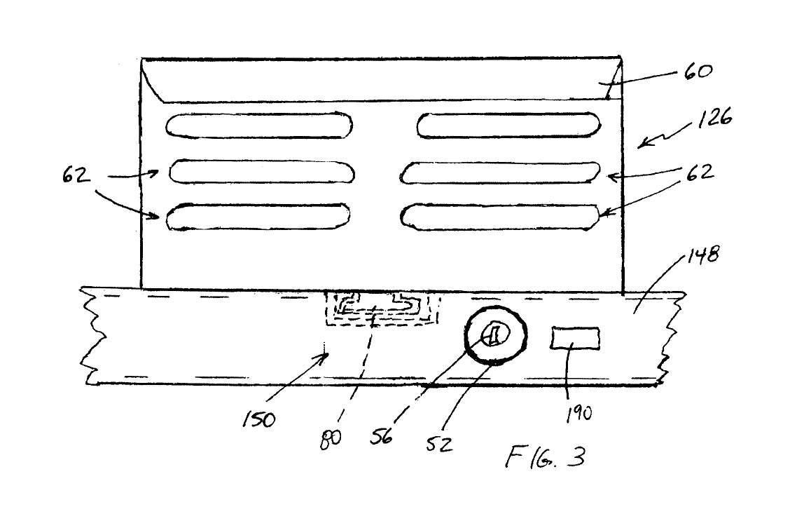

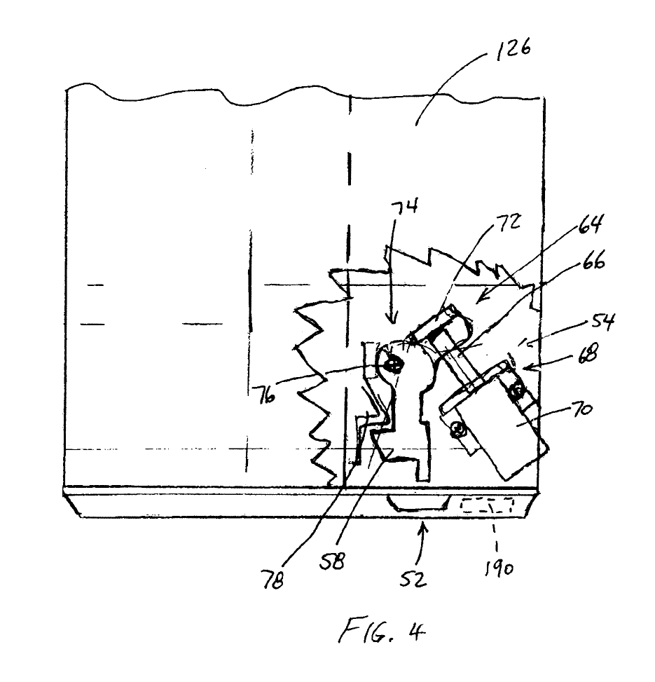

In one embodiment shown in FIG. 3, a drawer 126 is supported on a frame member 148 and is movable relative to the frame number 148 on a slide assembly 150 shown in phantom in FIG. 3. A key operated locking mechanism 52 is a mechanical override of an electrically actuated locking mechanism 54 shown in FIG. 4. The mechanism 52 includes a key receiver 56 which allows a user to use a key to actuate a latch 58 shown in FIG. 4. The drawer 126 is formed to include a handle 60 and a number of perforations 62. The perforations 62 are formed to allow airflow into the drawer 126 when the drawer is positioned in the cabinet 10 of the refrigerator 40 to thereby allow the drawer 126 to be maintained at the desired temperature. The locking mechanism 54 shown in FIG. 4 includes an electrically actuated lever 64 which is pushed by a rod 66 of a solenoid 68. The solenoid 68 includes a spring bias member (not shown) inside of a housing 70. The spring bias member urges the rod 66 to retract into the housing 70. The rod 66 is engaged with a tab 72 of the locking mechanism 54 so that the rod 66 pulls on the tab 70 to cause the lock 74 to pivot about a pin 76 such that the latch 58 engages a pawl 78 coupled to a slide 80 secured to the bottom of the drawer 126. The solenoid 68 may be electrically energized to extend the rod 66 causing the lock 74 to pivot about the pin 76 so that the latch 58 disengages from the pawl 78. Thus, the solenoid acts to permit the drawer 126 to be accessed by unlatching the locking mechanism 54. The mechanism 52 overcomes the spring bias to move the latch 58 out of engagement with the pawl 78. In this manner, the locking mechanism 54 operates as an access control device similar to the access control device 28 described with regard to FIG. 1.

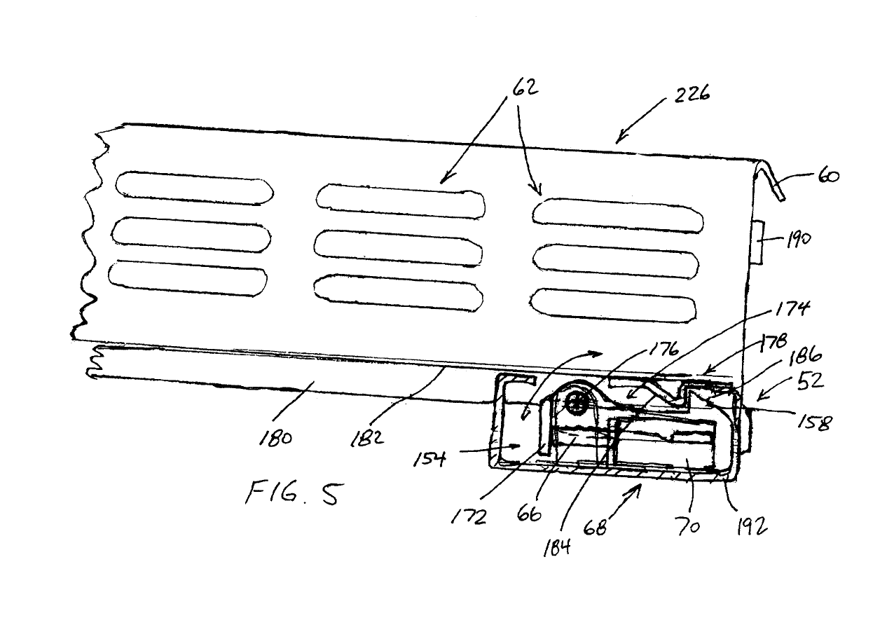

In another embodiment shown in FIG. 5, a drawer 226 is shown with perforations 62 formed in the side of the drawer 226 to allow airflow in turn to promote acceptable temperature uniformity within the usable areas of the storage space. The drawer 226 moves on a slide 180 with a pawl 178 coupled to the drawer bottom 182. In the illustrative embodiment of FIG. 5, a locking mechanism 154 includes a lock 174 that is formed to include a tab 172 which is coupled to a rod 66 of a solenoid 68. The lock 174 is rotatable about a pin 176 and includes a latch 158 which engages the pawl 178. The spring bias member (not shown) of the solenoid 68 urges the rod 66 to retract into a housing 70 of the solenoid 68. When the solenoid 68 is electrically energized, the rod 66 is urged outwardly to cause the lock 174 to rotate about the pin 176 to disengage a latch 158 from the pawl 178. A lower surface 184 of the pawl engages an upper surface 186 of the lock when the drawer is moved to a closed position. Engagement of the surface 184 with the surface 186 causes the spring bias member to be overcome such that the lock 174 rotates about pin 176 to move the latch 158 downwardly allowing the pawl 178 to pass over the latch 158. Once the pawl 178 is clear of the latch 158, the spring bias member acts on the rod 66, urging the lock 174 to rotate into the locked position shown in FIG. 5.

Each of the drawers 26, 126, 226 may include an indicator 190 as shown in FIGS. 3-5. The indicator 190 in the illustrative embodiment of FIGS. 3-5 is a light which is illuminated when the solenoid 68 is energized permitting the drawer 126, 226 to be accessed. The indicator 190 provides a visual indication to a user of the location of the inventory to be accessed. In the illustrative embodiments, the indicator 190 is a light. It should be understood that in some embodiments the indicator may be a mechanical indicator attached to either the lock 74 or lock 174. For example, an indicator may be attached to the respective locks 74, 174 so that movement of the lock 174 or the lock 74 about the respective pins 176 or 76 cause a colored indicator to appear in an aperture formed in the rail 148 of the embodiments of FIGS. 3-4. Similarly, a housing 192 that encloses the locking mechanism 154 shown in FIG. 5 may be formed to include an aperture through which an indicator is visible when the lock 174 is pivoted to an unlocked position.

In some embodiments, each door 20, or drawer 26, 126, 226 will include a sensor similar to the sensor 22 shown in FIG. 1. The sensor 22 is operable to detect that the door 20 is in an open position. The controller 18 is operable to determine when the signal from the sensor 22 indicates an open condition at an inappropriate time, such as when access has not been permitted to a particular storage location. The controller 18 may then log a note indicating an improper access, or may signal an external alarm indicating that unauthorized access has been made to a portion of the refrigerator 40. The alarm signal may also be forwarded to the system 34 to be acted on by a person at a central location.

In some embodiments, refrigerator 40 may further include radio frequency identification (RFID) capabilities which allow the controller 18 to detect a particular item of inventory by sensing a RFID signal from a particular article of inventory. This allows the refrigerator 40, through the controller 18, to positively monitor inventory stored in the refrigerator 40. A user may “check out” an item of inventory by accessing the particular inventory location as described above and scanning the item of inventory to be removed. If an item of inventory is removed unexpectedly or without being scanned, the controller 18 would note the time of removal of the particular item of inventory in the log so that the removal could be associated with the user who accessed the inventory location.

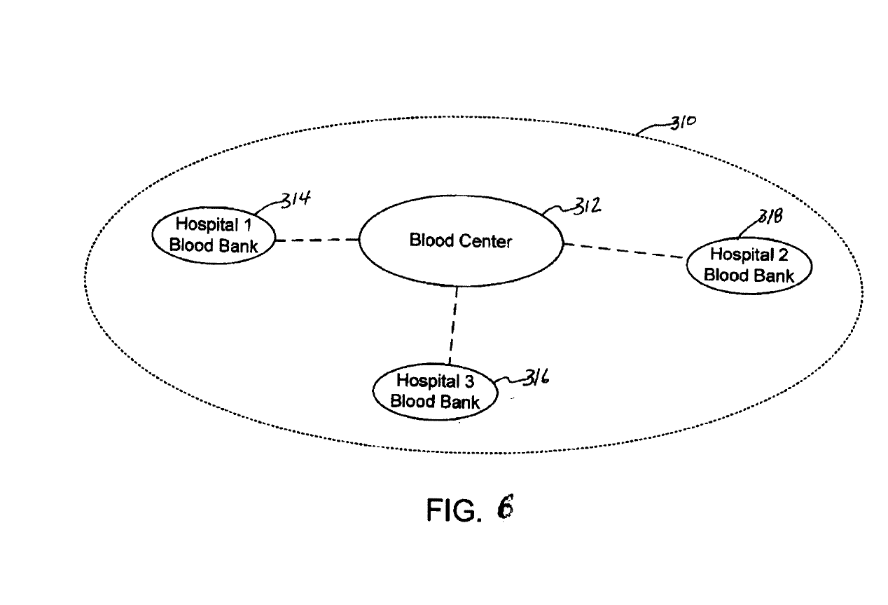

In another implementation, an inventory and quality control system for medical products 310 is illustratively embodied as a platelet control system 310 as shown in FIG. 6. System 310 comprises a central control operation embodied as a blood center 312. Multiple point-of-use facilities are embodied as hospital blood banks 314, 316 and 318. Each hospital blood bank 314, 316 or 318 functions as an independent inventory and quality control operation. However, each hospital blood bank 314, 316, 318 is in communication with the blood center 312 to share data with the blood center 312 regarding the platelet inventory present at the hospital blood bank 314, 316, 318 as well as sharing data related to the consumption of platelets at the particular hospital blood bank 314, 316, 318.

Platelets require various operational parameters to be controlled in order to maintain the platelets in a usable condition. For example, platelets require agitation to prevent the platelets from coagulating. In addition, the temperature of the platelets must be maintained within a particular range for proper preservation. Even under appropriate conditions, platelets have a limited shelf life and upon reaching the end of the shelf life, the platelets are not acceptable for clinical use and must be discarded.

Appropriate inventory management across the entire system 310 requires access to consumption data, expiration data, and current inventory. In some cases, additional data such as donor identification, origin location, processing lot information, or blood type may also be tracked. Control system 310 gathers appropriate data from hospital blood banks 314, 316, 318 to populate system wide data and processes the data to determine where platelet inventory should be maintained in the system 310 to minimize the loss of platelets due to expiration. If too much inventory is maintained at a particular hospital blood bank 314, 316 or 318, platelets may expire due to exceeding the shelf life. To the extent that those platelets may have been consumed at other hospital blood banks, there is an economic loss.

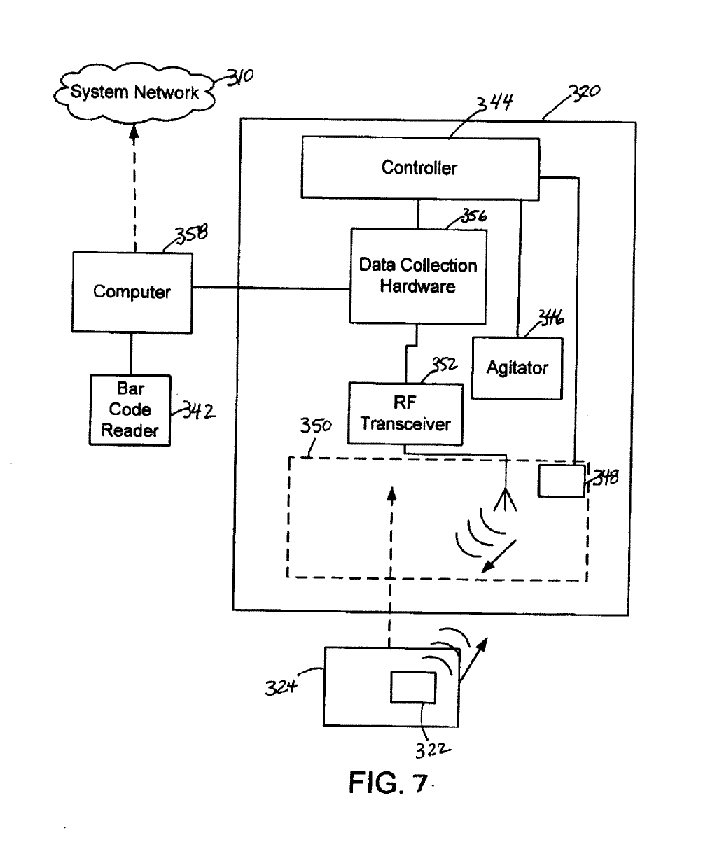

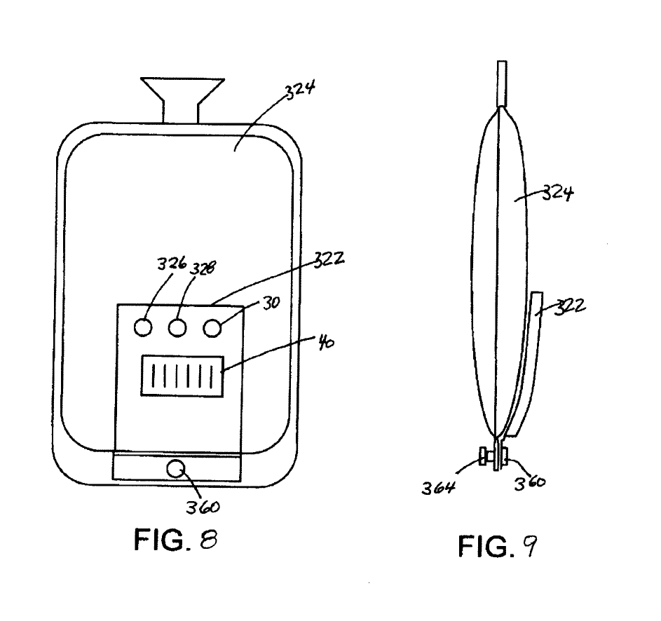

Platelets which are not stored properly may also become unusable. In the system 310, storage conditions data is associated with inventory data to establish that sufficient quality control has been maintained to keep the platelets in the usable inventory pool. According to the present disclosure, a storage unit embodied as a platelet incubator 320 shown in FIG. 7 cooperates with a radio frequency (RF) enabled tag 322 coupled to a platelet bag 324 to monitor the inventory and quality control characteristics of the platelet bag 324. Additionally, the tag 322 is configured to receive RF signals from the incubator 320 to update a status of the platelet bag 324 in the RF tag 322. The RF tag 322 includes a number of indicators which provide a visual indication of the status of the bag 324 to a user.

Referring to FIG. 8, the RF tag 322 includes a green light emitting diode (LED) 326 which is illuminated when the status of the contents of the bag 324 to which the tag 322 is attached is in a usable condition. Tag 322 also includes a yellow LED 328 which is illuminated when the contents of the associated bag 324 is near the end of the acceptable shelf life. In the illustrative embodiment, the yellow LED 328 is illuminated when the platelets in bag 324 are within 324 hours of expiration. Tag 322 also includes a red LED 330 which is illuminated when the platelets in the associated bag 330 are not acceptable for use. For example, the red LED 330 is illuminated if the platelets have reached the end of their shelf life or have been exposed to improper storage conditions. When a user has a need for a blood product, an inventory transaction is processed by system 310 to remove a particular bag 324 from the storage in the incubator 320. System 310 communicates with incubator 320 which in turn provides a signal to the tag 322 associated with the particular bag 324 that the bag 324 is to be picked from inventory. Upon receipt of the pick signal, tag 322 flashes the green LED 326 to indicate to the user the particular bag 324 to be selected. The user then selects the bag 324 and cross-checks with the system 310 to confirm the appropriate bag 324 has been selected. This reduces the potential for an improper bag 324 from being pulled from inventory.

As shown in FIG. 13, tag 322 includes a processor 332, a memory 334 coupled to processor 332, an RF transceiver 336, and a battery 338. Processor 332 is operable to control RF communications and to process associated data to control the operation of the LED's 326, 328, and 330. Each tag 322 further includes generic bar code label 340. Label 340 permits a user to employ a bar code reader 342 in communication with system 310 to identify the associated bag 324 to the system 310 when a RF reader is not available.

The tag 322 also includes a thermocouple 360 coupled to the processor 332 and configured to sense a temperature experienced by the tag 322 and bag 324. The processor 332 logs the temperature data in memory 334. The logged data is wirelessly transmitted to the system 310 for quality control logging.

Referring again now to FIG. 7, as suitable incubator for incubator 320 is described in U.S. patent application Ser. No. 310/979,391 titled “PLATELET INCUBATOR” filed Nov. 2, 2004 which is incorporated herein by reference. Incubator 320 includes a controller 344 which receives information from an agitator 346 regarding the operation of the agitator 346. In addition, controller 344 is coupled to a temperature sensor 348 which is operable to measure the temperature within a storage space 350 of the incubator 320. In the illustrative embodiment of the present disclosure, incubator 320 further includes an RF transceiver 352 which is coupled to an antenna 354 positioned adjacent to the storage space 350 and operable to communicate with tags 322 coupled two bags 324 positioned in the storage space 350. RF transceiver 352 is coupled to data collection circuitry 356 which communicates with the controller 344 of the incubator 320 and an external computer 358 which is in communication with the system 310. In some embodiments, the RF transceiver 352 and antenna 354 are configured to continuously communicate with all of the tags 322 present in the storage space 350. In other embodiments, the RF transceiver 352 and antenna 354 are positioned to communicate with the tags 322 as they are inserted or removed from the storage space 350. In some embodiments, multiple RF transceivers 352 and antennae 354 are positioned in various locations within the storage space 350 each combination of transceiver and antenna 354 associated with a different storage location within the storage space 350 such that the system 310 can associate the appropriate storage location with each tag 322.

Circuit 356 is operable to gather data from controller 344 related to the storage conditions of the incubator 320 and to associate that data with the inventory data gathered from the tags 322 in the storage space 350. This associated data is communicated to the external computer 358 to create a record for each bag 324 which confirms that the bag 324 has been properly stored. Associated data for a particular hospital blood bank 314, 316, 318 is communicated to blood center 312 where the system 310 processes the system-wide inventory data to manage inventory levels at the various locations throughout the system 310.

Under certain conditions controller 344 of the incubator 320 may form an error message related to the operation of the incubator 320 which would compromise the quality of the platelets stored in the incubator 320. If such an error were to occur, circuit 356 would communicate the failure to be external computer 358 and thereby the system 310. If the system 310 determines that the failure has rendered the platelets stored in the incubator 320 to be unusable, then system 310 would communicate the change in status to the tags 322 attached to the bags 324 of the unusable platelets such that the tags 322 would illuminate the red LED 330 on each bag 324 to indicate that the platelets are unusable. The status of the bags 324 may also change based on the temperature data acquired by the thermocouple 360. Thus, each bag 324 may be monitored individually by the associated tag 322 or as a group by the incubator 320. While the tag 322 of the illustrative embodiment includes a sensor embodied as a thermocouple 360, it should be understood that any of a number of sensors may be coupled to processor 332 and monitor the conditions experienced by the associated bag 324. For example, in some embodiments the tag 322 may further comprise a sensor, such as an accelerometer, to sense the motion of the sensor 322 and associated bag 324. By maintaining a continuous record of the storage conditions of the bags 324, system 310 can maintain appropriate inventory records and quality control records of the platelets stored throughout the system 310. This approach provides for an automated maintenance of inventory and quality control records to assist the various entities in system 310 with meeting regulatory compliance requirements.

Tags 322 are reusable when the contents of an associated bag 324 are consumed. Tags 322 are tamper-resistant and if a tag 322 is removed from a bag 324, the tag 322 goes into an error mode in which none of the LEDs 326, 328, 330 are illuminated. Each tag 322 has a unique serial number which is communicated through the RF signal and is associated with the barcode label 340. System 310 is capable of having multiple records associated with each tag 322, but the system 310 will create a separate record each time the tag 322 is used based on the date and time the tag 322 is associated with the system 310.

In one embodiment shown in FIGS. 8 and 9, tag 322 is secured to a bag 324 via a coupler 360 which when connected signals the tag 322 that the tag 322 is a fixed to a bag 324. Once tag 322 is a fix to a bag 324, a data record for the information associated with the bag 324 is permitted to be associated with the tag 322 by the system 310. Removal of the coupler 360 causes a data record to be written to system 310 that tag 322 has been removed from bag 324 and tag 322 will go into the error mode in which all of the LEDs 326, 328, 330 are turned off.

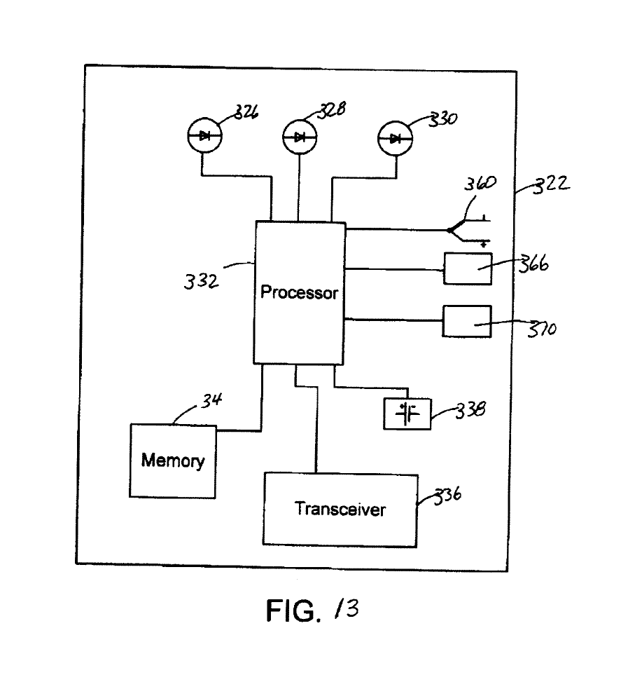

In FIG. 12, tag 322 is shown to include two electrical contacts 366 and 370 which are each in communication with processor 332. When the circuit between contacts 366 and 370 is closed, a signal is communicated to the processor 332 that the tag 322 is attached to a bag 324. Referring now to FIG. 9, tag 322 is shown with a pin 364 of coupler 360 in an engaged position such that electrical contact 366 and an electrical contact 370 are each contacted so that a circuit therebetween is made and a signal that the coupler 360 is connected is communicated to processor 332. If circuit 366 is broken, tag 322 goes into error mode.

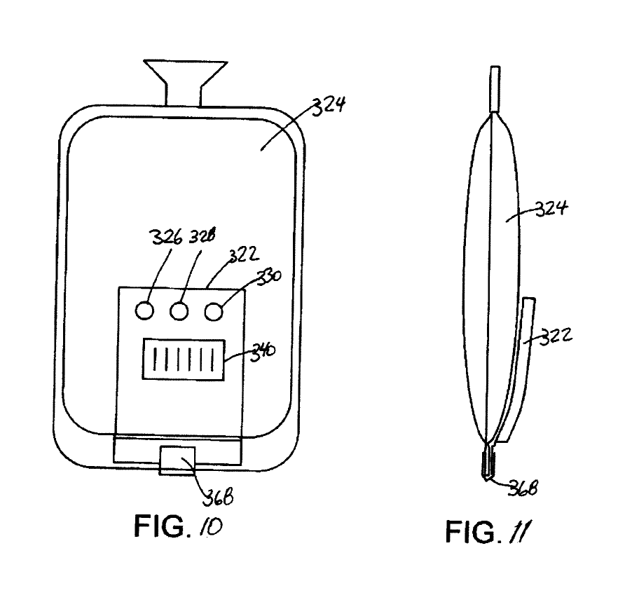

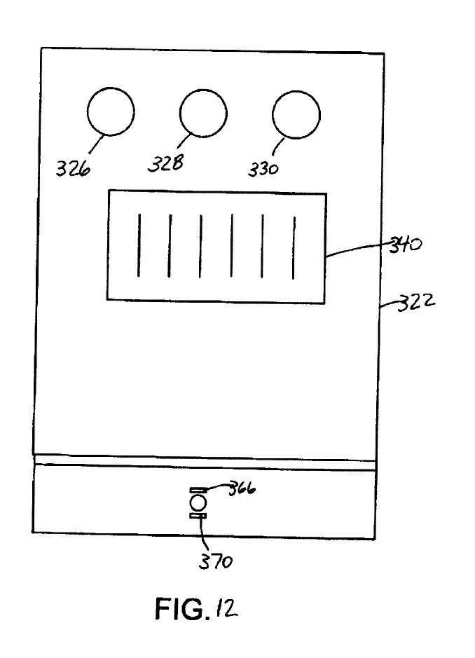

In an alternative embodiment shown in FIGS. 10 and 11, a coupler 368 is embodied as a clip which secures tag 322 to bag 324. Coupler 368 provides the connection between contacts 366 and 370 and thereby closes the circuit to communicate that the coupler 368 is in place. If coupler 368 is removed, circuit 366 is broken and tag 322 goes into error mode.

System 310 is capable of monitoring the elapsed time a particular bag 324 is away from system 310 and thereby outside of the acceptable storage conditions. A bag 324 may be re-associated with system 310 after having been checked out if the elapsed time away from system 310 is within acceptable range. Also, system 310 is capable of monitoring accumulated time away from system 310 such the bag that is checked out multiple times and not used may be marked as unusable if the cumulative time away from system 310 exceeds an acceptable amount.

Although certain illustrative embodiments have been described in detail above, variations and modifications exist within the scope and spirit of this disclosure as described and as defined in the following claims.

1. A climate controlled storage device for medical products comprising
a plurality of storage locations,
an access control device associated with each storage location,
a controller monitoring the operational parameters of the climate controlled storage device and controlling the climate of the storage locations, and
an interface coupled to the controller and the access control devices, the interface receiving signals from the controller to permit access to one of the plurality of storage locations and controlling the access control device associate with the one of the plurality of storage locations to permit a user to access said storage location.

2. The climate controlled storage device of claim 1, wherein the access control device is electrically actuated.

3. The climate controlled storage device of claim 2, wherein the controller permits access to the storage locations based on a user input.

4. The climate controlled storage device of claim 3, wherein the controller logs the access to the storage location.

5. The climate controlled storage device of claim 2, wherein the controller maintains a record of inventory stored in the climate controlled storage device.

6. The climate controlled storage device of claim 2, wherein the inventory records associate a particular item of inventory with a particular storage location.

7. The climate controlled storage device of claim 1, wherein the controller communicates data to an external inventory and access control monitoring system or a user via at least one of a text message to cellular device, phone message, or computer via network.

8. The climate controlled storage device of claim 1, wherein the controller monitors the status of the plurality of storage locations and logs unauthorized access to a particular storage location.

9. The climate controlled storage device of claim 1, wherein an access control device further includes an indicator that identifies the storage location to be accessed when the access control device is actuated to permit access to the storage location.

10. The climate controlled storage device of claim 1, wherein a first storage location may be contained within a second storage location such that a first access control device permits access to the second storage location and a second access control device permits access to the first storage location.

11. The climate controlled storage device of claim 1, wherein the controller prevents access to a storage location if the inventory item stored in the storage location is unusable due to age or improper climate conditions for storage.

12. The climate controlled storage device of claim 1, wherein an access control device is a solenoid operated locking mechanism.

13. The climate controlled storage device of claim 12, wherein the access control device includes a latch.

14. The climate controlled storage device of claim 13, wherein the latch is biased to a closed position.

15. The climate controlled storage device of claim 1, wherein the storage locations permit air flow therethrough to permit the climate to be maintained throughout the storage device.

16. A medical products inventory and quality control system comprising
a plurality of medical products storage containers, each container including a marking tag configured to transmit and receive wireless signals indicative of parameters associated with the medical products stored therein; and
a medical products storage device including (i) a storage space, (ii) a controller configured to monitor the operational parameters of the medical products storage device, (iii) a wireless transceiver configured to communicate with the tags of blood product storage containers positioned in the storage space, (iv) a data processing circuit in communication with the controller and the wireless transceiver, the data processing circuit operable to associate the operational parameters of the medical products storage device with the parameters of the medical products stored in each container to create an inventory and quality control record for each container.

17. The system of claim 16, wherein the system further comprises a computer in communication with the data processing circuit, the computer operable to display the inventory and quality control record for each container.

18. The system of claim 16, wherein the system further includes a plurality of computers in a network, each computer in communication with the data processing circuit of at least one medical products storage device, the network configured to gather inventory and quality control records from a plurality of medical products storage devices.

19. The system of claim 16, wherein the marking tag comprises a processor, a memory device coupled to the processor, an RF transceiver coupled to the processor, and a battery coupled to the processor.

20. The system of claim 16, wherein the tag further comprises an indicator configured to indicate a status of the contents of the storage container associated with the tag.
